(19) United States
(12) Patent Application Publication
(10) Pub. No.: US 2024/0141349 A1
(43) Pub. Date: May 2, 2024

(54) USING siRNAs AGAINST TAU CIRCULAR RNAS AS A RATIONAL THERAPY FOR ALZHEIMER'S DISEASE

(71) Applicant: University of Kentucky Research Foundation, Lexington, KY (US)

(72) Inventors: Stefan Stamm, Lexington, KY (US); Justin Ralph Welden, Lexington, KY (US)

(21) Appl. No.: 18/498,834

(22) Filed: Oct. 31, 2023

Related U.S. Application Data
(60) Provisional application No. 63/420,923, filed on Oct. 31, 2022.

(51) Int. Cl. C12N 15/113

(52) U.S. Cl. CPC C12N 15/113; C12N 2310/11; C12N 2310/53

(57) ABSTRACT

The present disclosure concerns isolated double stranded (ds) silencing RNA (siRNA) to target a backsplice junction between exons 12 and 7 in the MAPT gene. The ds siRNA include at least one nucleotide from the 3′ terminus of exon 12 linked to at least one nucleotide of the 5′ terminus of exon 7, thereby overlapping the backsplice junction. Targeting the junction ensure that that ds siRNA are limited in targeting the circular RNA produced by the backsplicing while allowing normal expression of the MAPT gene.

### CROSS-REFERENCE TO RELATED APPLICATIONS

This application claims priority to U.S. Provisional Patent Application 63/420,923, filed Oct. 31, 2022, the content of which is hereby incorporated by reference in its entirety.

### GOVERNMENT SUPPORT

This invention was made with support from grant 1 R41 AG078096-01 from the STTR Program at the National Institute of Health. The government may have rights to the invention.

### SEQUENCE LISTING

This application includes a sequence listing filed concurrently herewith as file UKY2669PA.xml created 2023-10-31 and having a file size of 33.4 kB.

### BACKGROUND

The formation of neurofibrillary tangles (NFTs) is a hallmark of Alzheimer's disease and related tauopathies, such as frontotemporal lobar degenerations (FTLDs) and Frontotemporal dementia with Parkinsonism linked to Chromosome 17 (FTDP-17), now called frontotemporal lobar degeneration (FTPD-Tau). NFTs are composed of aggregates of microtubule associated protein tau (MAPT), which is usually hyperphosphorylated.

Alzheimer's disease (AD) affects about 10% of people older than 65 and for 2020 generated costs of $355 billion with $18 billion for “treatments.” In AD, neurofibrillary tangles (NFTs) formed by tau proteins cause neuronal death and disease progression. There remains, however, no rational therapy for Neurofibrillary tangle formation available. Interestingly, Neurofibrillary tangles also form after traumatic brain injury and in rare genetic forms (FTLD-tau (frontotemporal lobar degeneration)). There is currently no animal model for NFT formation.

Current approaches for therapy focus on the presence of beta-amyloid, with antibodies against beta amyloid Aducanumab being FDA approved and Lecanemab in phase III clinical trial. There are no therapies against NFTs or the tau proteins that aggregate to form the same, though their targeting presents a potential route of therapy.

### SUMMARY

A 1^{st} aspect of the present disclosure, either alone or in combination with any other aspect herein, concerns an isolated double stranded (ds) silencing ribonucleic acid (siRNA) comprising a nucleotide sequence comprised of a 3′ terminal nucleic acid from exon 12 of the MAPT gene fused to a 5′ terminal nucleic acid from either exon 7 of the MAPT gene or exon 10 of the MAPT gene.

A 2^{nd} aspect of the present disclosure, either alone or in combination with any other aspect herein, concerns the isolated ds siRNA of the 1^{st} aspect, wherein the nucleotide sequence comprises 21 nucleotides in length, with up to 20 nucleotides being derived from the 3′ end of exon 12 of the MAPT gene.

A 3^{rd} aspect of the present disclosure, either alone or in combination with any other aspect herein, concerns the isolated ds siRNA of the 1^{st} aspect, wherein the nucleotide sequence comprises 21 nucleotides in length, with up to 20 nucleotides being derived from the 5′ end of exon 7 of the MAPT gene.

A 4^{th} aspect of the present disclosure, either alone or in combination with any other aspect herein, concerns the isolated ds siRNA of the 1^{st} aspect, wherein the nucleotide sequence comprises 21 nucleotides in length, with up to 20 nucleotides being derived from the 5′ end of exon 10 of the MAPT gene.

A 5^{th} aspect of the present disclosure, either alone or in combination with any other aspect herein, concerns the isolated ds siRNA of the 1^{st} aspect, wherein the nucleotide sequence comprises 21 contiguous nucleotides in length from SEQ ID NO: 28.

A 6^{th} aspect of the present disclosure, either alone or in combination with any other aspect herein, concerns the isolated ds siRNA of the 5^{th} aspect, wherein the nucleotide sequence comprises a sequence selected from SEQ ID NOs: 8-19 and 29-36.

A 7^{th} aspect of the present disclosure, either alone or in combination with any other aspect herein, concerns the isolated ds siRNA of the 1^{st} aspect, wherein the wherein one or more nucleic acids are modified.

An 8^{th} aspect of the present disclosure, either alone or in combination with any other aspect herein, concerns the isolated ds siRNA of the 7^{th} aspect, wherein the modification includes incorporation of 2′-O-methylation, 2′-O-ethylation, 2′-fluorination, 2′-desoxylation, and/or 5′-phosphorylation, mono-phosphothionate, or di-phosphothionate.

A 9^{th} aspect of the present disclosure, either alone or in combination with any other aspect herein, concerns the isolated ds siRNA of the 1st aspect, wherein the double strands are two separate annealed strands.

A 10^{th} aspect of the present disclosure, either alone or in combination with any other aspect herein, concerns the isolated ds siRNA of the 1^{st} aspect, wherein the double strands are a self-annealed single strand.

An 11^{th} aspect of the present disclosure, either alone or in combination with any other aspect herein, concerns the isolated ds siRNA of the 10^{th} aspect, further comprising a spacer of 3 to about 20 unmatching nucleotides in length between a sense portion and an antisense portion.

A 12^{th} aspect of the present disclosure, either alone or in combination with any other aspect herein, concerns the isolated ds siRNA of the 1^{st} aspect, wherein the double strands are a self-annealed single strand.

A 13^{th} aspect of the present disclosure, either alone or in combination with any other aspect herein, concerns a lipid nanoparticle comprising the isolated ds siRNA of the 1^{st} aspect and a lipid membrane.

A 14^{th} aspect of the present disclosure, either alone or in combination with any other aspect herein, concerns a method of treating or alleviating formation of neurofibrillary tangles comprising administering the isolated ds siRNA of the 1st aspect to a subject.

A 15^{th} aspect of the present disclosure, either alone or in combination with any other aspect herein, concerns the method of the 14^{th} aspect, wherein the ds siRNA is administered nasally and/or by intrathecal injection.

A 16^{th} aspect of the present disclosure, either alone or in combination with any other aspect herein, concerns a method to treat aggregates of MAPT proteins in brain tissue, comprising administering the isolated ds siRNA of the 1^{st} aspect to a subject.

A 17^{th} aspect of the present disclosure, either alone or in combination with any other aspect herein, concerns the method of the 16^{th} aspect, wherein the ds siRNA is administered nasally and/or by intrathecal injection.

An 18^{th} aspect of the present disclosure, either alone or in combination with any other aspect herein, concerns a method for alleviating Alzheimer's Disease (AD) comprising administering to a subject with AD the isolated ds siRNA of the 1^{st} aspect.

A 19^{th} aspect of the present disclosure, either alone or in combination with any other aspect herein, concerns a method for treating AD comprising administering to a subject with AD the isolated ds siRNA of the 1^{st} aspect.

### BRIEF DESCRIPTION OF THE DRAWINGS

FIG. 1 shows the backsplicing phenomenon in the MAPT gene for the 12->7 circRNA, with A>I editing increasing translation thereof and consequently increasing aggregates of linear and circular MAPT proteins.

FIG. 2 shows linear tau and circular tau circular proteins assembled in vitro and visualized by TEM. Aggregation was initiated through heparin for linear tau for isoform 441 and by yeast tRNA with the other samples. Scale bar is 200 nm.

FIG. 3 shows inhibition of the 12->7 and 12->10 circRNA. A shows inclusion of the FLAG in exon 7. B shows western blotting with anti-FLAG or anti-Tau antibodies following doxorubicin induction in the mouse model. C shows siRNA concentrations plotted against circRNA concentration. D shows inclusion of the FLAG in exon 10. E shows western blotting with anti-FLAG or anti-Tau antibodies following doxorubicin induction in the mouse model. F shows siRNA concentrations plotted against circRNA concentration.

FIG. 4 shows induction of circRNA expression in a doxorubicin treated mouse followed by FLAG immunoprecipitation and anti-FLAG western blot.

FIG. 5 shows brain tissue localization of the circRNA in a dissected induced mouse.

FIG. 6 shows the correlation between the Braak stage and the increase in circRNA.

FIG. 7 shows a model for A>I editing and the resulting circular proteins with a first and second round of translation.

FIG. 8 shows western blotting for circRNA in the mouse model following treatment with the indicated concentrations of siRNA.

FIG. 9 shows measured circRNA following administration of the identified concentrations of siRNA.

FIG. 10 shows an overview of sequence optimization of the siRNA.

### DESCRIPTION

Scientific and technical terms used herein are intended to have the meanings commonly understood by those of ordinary skill in the art. Such terms are found defined and used in context in various standard references illustratively including M. R. Green and J. Sambrook, Molecular Cloning: A Laboratory Manual, Cold Spring Harbor Laboratory Press; 4^{th} Ed., 2012; F. M. Ausubel, Ed., Short Protocols in Molecular Biology, Current Protocols; 5^{th} Ed., 2002; B. Alberts et al., Molecular Biology of the Cell, 4^{th} Ed., Garland, 2002; CRISPR/Cas: A Laboratory Manual, Doudna and Mali (eds), Cold Spring Harbor Laboratory Press, Cold Spring Harbor, NY, USA, 2016; D. L. Nelson and M. M. Cox, Lehninger Principles of Biochemistry, 4^{th} Ed., W. H. Freeman & Company, 2004; Herdewijn, P. (Ed.), Oligonucleotide Synthesis: Methods and Applications, Methods in Molecular Biology, Humana Press, 2004; Remington: The Science and Practice of Pharmacy, Lippincott Williams & Wilkins, 21st Ed., 2005; L. V. Allen, Jr. et al., Ansel's Pharmaceutical Dosage Forms and Drug Delivery Systems, 8^{th} Ed., Philadelphia, PA: Lippincott, Williams & Wilkins, 2004; and L. Brunton et al., Goodman & Gilman's The Pharmacological Basis of Therapeutics, McGraw-Hill Professional, 12^{th} Ed., 2011.

The singular terms “a,” “an,” and “the” are not intended to be limiting and include plural referents unless explicitly stated otherwise or the context clearly indicates otherwise.

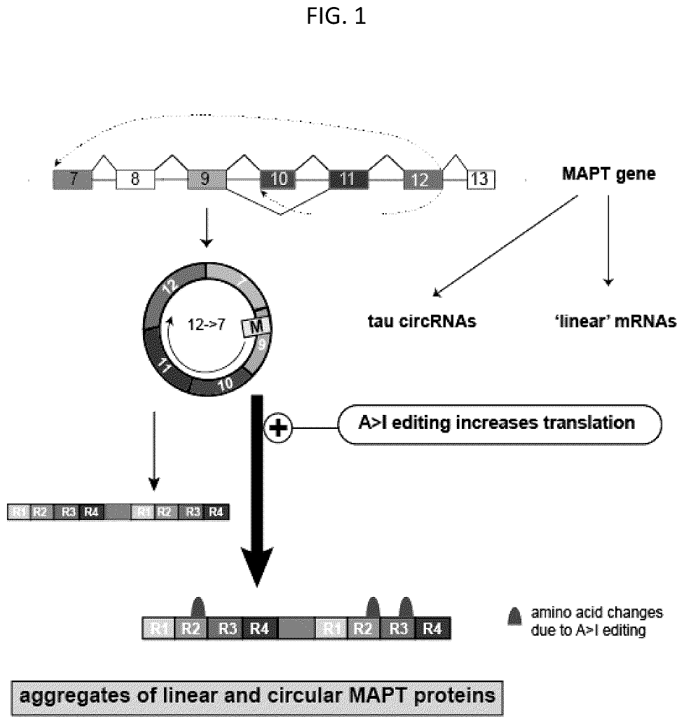

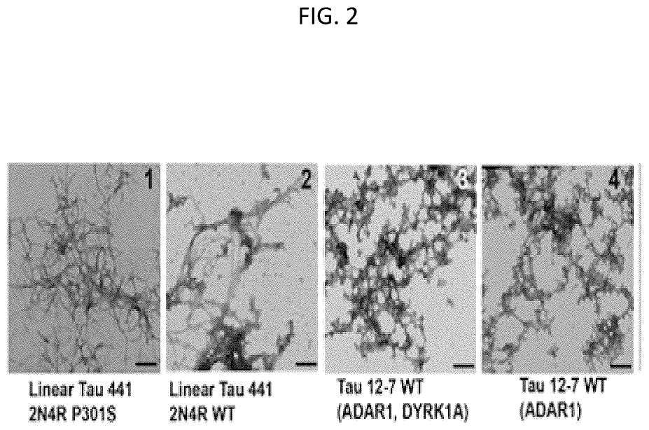

It has recently been identified that the MAPT gene will generate circular RNA molecules due to an identified backsplicing between exons 12 and 10 (12->10) and exons 12 and 7 (12->7) (see, Welden JR et al.; Biochim Biophys Acta. 2018; 1864(9 Pt B):2753-60). Both backsplicing occurrences generate circular RNA (circRNA) molecules with 12->7 having a sequence length of 681 nucleotides (nt) encoding 227 amino acids and 12->10 having a sequence length of 288 nt, encoding 96 amino acids. The 12->7 circRNA features a natural start codon. The 12->10 circRNA has no native start codon, but two identified human mutations, V337M and K317M, will introduce in frame start codons. The translation of the circular RNAs is further feasible following an epigenetic modification of adenosine to inosine. The translated proteins cause the formation of neurofibrillary tangles from the linear tau proteins. Adenosine to inosine RNA editing of the 12->10 circRNA also causes translation, by generating AUI start codons from AUA codons. (I: Inosine). An adenosine in the AUA codon #99 is converted to inosine, generating an AUI start codon. FIG. 1 shows an overview of the backsplicing phenomenon. FIG. 2 confirms the expression of the cirRNAs for the 12->7 backsplice variants.

These circular tau RNA molecules allowed for the identification of human-specific tau circular RNAs which provide a target for AD and related neurodegenerative conditions. It is therefore an aspect of the instant disclosure to use silencing techniques, such as siRNA, to remove circular tau RNAs and cease protein translation thereof.

In some aspects, the present disclosure concerns isolated double stranded (ds) silencing ribonucleic acid (siRNA) to silence expression of a backsplice circRNA. In some aspects, the siRNA target one or more exon junctions within the circRNA. In some aspects, the siRNA target the backsplice junction where exon 12 is fused to exon 10 or 7, such as an siRNA targeting the 12->10 and or 12->7 junctions and the resulting circRNA therefrom. In some aspects, the siRNA include a terminal 3′ nucleic acid from exon 12 and an initial 5′ nucleic acid from exon 7 or exon 10. In some aspects, the siRNA include a G-G sequence to cover the 12->7 junction. In some aspects, the siRNA can include up to 20 additional contiguous nucleic acids of the 3′ terminus of exon 12, including 19, 18, 17, 16, 15, 14, 13, 12, 11, 10, 9, 8, 7, 6, 5, 4, 3, 2, and 1 contiguous nucleic acids. In some aspects, the siRNA can include up to 20 additional contiguous nucleic acids of the 5′ terminus of exon 7, including 19, 18, 17, 16, 15, 14, 13, 12, 11, 10, 9, 8, 7, 6, 5, 4, 3, 2, and 1 contiguous nucleic acids.

In some aspects, the siRNA include a sequence of about 19-23, including 20, 21, and 22, nucleic acids that target the 12->7 circRNA. SEQ ID NO: 1 sets forth the circRNA of the 12->7 circRNA:
GGGGCTGATGGTAAAACGAAGATCGCCACACCGCGGGGAGCAGCCCCTCCAGGCCAGAAGGGCCAGG CCAACGCCACCAGGATTCCAGCAAAAACCCCGCCCGCTCCAAAGACACCACCCAGCTCTG|GTGAACCTC CAAAATCAGGGGATCGCAGCGGCTACAGCAGCCCCGGCTCCCCAGGCACTCCCGGCAGCCGCTCCCGC ACCCCGTCCCTTCCAACCCCACCCACCCGGGAGCCCAAGAAGGTGGCAGTGGTCCGTACTCCACCCAAG TCGCCGTCTTCCGCCAAGAGCCGCCTGCAGACAGCCCCCGTGCCCATGCCAGACCTGAAGAATGTCAA|GTCCAAGATCGGCTCCACTGAGAACCTGAAGCACCAGCCGGGAGGCGGGAAG|GTGCAGATAATTAAT AAGAAGCTGGATCTTAGCAACGTCCAGTCCAAGTGTGGCTCAAAGGATAATATCAAACACGTCCCGGGA GGCGGCAGTGTGCAAATAGTCTACAAACCAGTTGACCTGAGCAAGGTGACCTCCAAGTGTGGCTCATTA GGCAACATCCATCATAAACCAG|GAGGTGGCCAGGTGGAAGTAAAATCTGAGAAGCTTGACTTCAAGG ACAGAGTCCAGTCGAAGATTGGGTCCCTGGACAATATCACCCACGTCCCTGGCGGAGGAAATAAAAAG (SEQ ID NO: 1) (|marks the different exon boundaries in the sequence). Additionally, as set forth in the examples, the cirRNA may be further adapted to include a tag, such as a FLAG tag. SEQ ID NO: 2 sets forth an exemplary sequence that includes a FLAG encoding sequence that does not cause a frame shift for the circRNA:
GGGGCTGATGGTAAAACGAAG|GACTACAAAGACCATGACGGTGATTATAAAGATCATGACATCGATT ACAAGGATGACGATGACAAG|ATCGCCACACCGCGGGGAGCAGCCCCTCCAGGCCAGAAGGGCCAGG CCAACGCCACCAGGATTCCAGCAAAAACCCCGCCCGCTCCAAAGACACCACCCAGCTCTG|GTGAACCTC CAAAATCAGGGGATCGCAGCGGCTACAGCAGCCCCGGCTCCCCAGGCACTCCCGGCAGCCGCTCCCGC ACCCCGTCCCTTCCAACCCCACCCACCCGGGAGCCCAAGAAGGTGGCAGTGGTCCGTACTCCACCCAAG TCGCCGTCTTCCGCCAAGAGCCGCCTGCAGACAGCCCCCGTGCCCATGCCAGACCTGAAGAATGTCAA|GTCCAAGATCGGCTCCACTGAGAACCTGAAGCACCAGCCGGGAGGCGGGAAG|GTGCAGATAATTAAT AAGAAGCTGGATCTTAGCAACGTCCAGTCCAAGTGTGGCTCAAAGGATAATATCAAACACGTCCCGGGA GGCGGCAGTGTGCAAATAGTCTACAAACCAGTTGACCTGAGCAAGGTGACCTCCAAGTGTGGCTCATTA GGCAACATCCATCATAAACCAG|GAGGTGGCCAGGTGGAAGTAAAATCTGAGAAGCTTGACTTCAAGG ACAGAGTCCAGTCGAAGATTGGGTCCCTGGACAATATCACCCACGTCCCTGGCGGAGGAAATAAAAAG (SEQ ID NO: 2) (|marks the different exon boundaries in the sequence).

In some aspects, the siRNA include a sense and an antisense strand. As set forth herein, primarily presented are the sense strands for the siRNA. It will be appreciated that complementary base pairing provides for the sequences of any antisense strand, including cytosine with guanine and adenine with uracil/thymine. In some aspects, the nucleic acids described herein may be double stranded or single stranded. In some aspects, the double strands are two separate annealed strands. In other aspects, the double strands are a self-annealed single strand. It will further be appreciated that any sequence set forth herein may include one or more additional nucleic acids. In some aspects, each sense strand may include one or more additional nucleic acids at the 5′ and/or 3′ end on the sequence. It will further be appreciated that the corresponding antisense may also include one or more additional nucleic acids at the 5′ and/or 3′ end. In some aspects, the siRNA as set forth herein include a 5′ and/or 3′ overhang of one or more nucleic acids. Such may include 2 or more nucleic acids. The 5′ and/or 3′ overhang may be selected to avoid hybridization with the corresponding circRNA. For example, SEQ ID NO: 3 sets forth a sense strand for an siRNA targeting the 12->7 exon junction with a 3′ overhang shown in lower case: 5′AGGAAAUAAAAAGGGGGCUtt (SEQ ID NO: 3). Accordingly, the antisense strand can include SEQ ID NO: 4: 3′AGCCCCCUUUUUAUUUCCUcc 5′ (SEQ ID NO: 4).

In some aspects, the target sequence of the siRNA of the instant disclosure concerns the 12->7 exon junction. In some aspects, such as where the FLAG tag was introduced into exon 7, the target sequence includes: CTGGCGGAGGAAATAAAAAGGGGGCTGATGGTGACTACAA (SEQ ID NO: 7). Accordingly, siRNA sense strands may include at least one nucleic acid in exon 12 and one nucleic acid of exon 7. Table 1 sets forth exemplary sequences, their respective reference labels as used in the working examples and figures, and their respective SEQ ID NOs (SEQ ID NOs: 20-27 accordingly include some FLAG encoding nucleic acid sequence at the 3′ end).

TABLE 1

| SEQ | | Reference |
|---|---|---|
| ID NO | Sequence | Name |
| 8 | CTGGCGGAGGAAATAAAAAGG | 7_1 |
| 9 | TGGCGGAGGAAATAAAAAGGG | 7_2 |
| 10 | GGCGGAGGAAATAAAAAGGG | 7_3 |
| 11 | GCGGAGGAAATAAAAAGGGGG | 7_4 |
| 12 | CGGAGGAAATAAAAAGGGGG | 7_5 |
| 13 | GGAGGAAATAAAAAGGGGGCT | 7_6 |
| | | (or 7_jv3) |
| 14 | GAGGAAATAAAAAGGGGGCTG | 7_7 |
| 15 | AGGAAATAAAAAGGGGGCTGA | 7_8 |
| 16 | GGAAATAAAAAGGGGGCTGAT | 7_9 |
| 17 | GAAATAAAAAGGGGGCTGATG | 7_10 |
| 18 | AAATAAAAAGGGGGCTGATGG | 7_11 |
| 19 | AATAAAAAGGGGGCTGATGGT | 7_12 |
| 20 | ATAAAAAGGGGGCTGATGGTG | 7_13 |
| 21 | TAAAAAGGGGGCTGATGGTGA | 7_14 |
| 22 | AAAAAGGGGGCTGATGGTGAC | 7_15 |
| 23 | AAAAGGGGGCTGATGGTGACT | 7_16 |
| 24 | AAAGGGGGCTGATGGTGACTA | 7_17 |
| 25 | AAGGGGGCTGATGGTGACTAC | 7_18 |
| 26 | AGGGGGCTGATGGTGACTACA | 7_19 |
| 27 | GGGGGCTGATGGTGACTACAA | 7_20 |

In some aspects, the target sequence includes: CTGGCGGAGGAAATAAAAAGGGGGCTGATGGTAAAACGAA (SEQ ID NO: 28). siRNA sense strands may include at least one nucleic acid in exon 12 and one nucleic acid of exon 7. Table 2 sets forth exemplary sequences, their respective labels, and their respective SEQ ID NOs.

TABLE 2

| SEQ | | Reference |
|---|---|---|
| ID NO | Sequence | Name |
| 8 | CTGGCGGAGGAAATAAAAAGG | 7_1 |
| 9 | TGGCGGAGGAAATAAAAAGGG | 7_2 |
| 10 | GGCGGAGGAAATAAAAAGGG | 7_3 |
| 11 | GCGGAGGAAATAAAAAGGGGG | 7_4 |
| 12 | CGGAGGAAATAAAAAGGGGG | 7_5 |
| 13 | GGAGGAAATAAAAAGGGGGCT | 7_6 |
| | | (or 7_jv3) |
| 14 | GAGGAAATAAAAAGGGGGCTG | 7_7 |
| 15 | AGGAAATAAAAAGGGGGCTGA | 7_8 |
| 16 | GGAAATAAAAAGGGGGCTGAT | 7_9 |
| 17 | GAAATAAAAAGGGGGCTGATG | 7_10 |
| 18 | AAATAAAAAGGGGGCTGATGG | 7_11 |
| 19 | AATAAAAAGGGGGCTGATGGT | 7_12 |
| 29 | ATAAAAAGGGGGCTGATGGTA | 7_21 |
| 30 | AAAAAGGGGGCTGATGGTAA | 7_22 |
| 31 | AAAAGGGGGCTGATGGTGAAA | 7_23 |
| 32 | AAAGGGGGCTGATGGTGAAAA | 7_24 |
| 33 | AAGGGGGCTGATGGTGAAAAC | 7_25 |
| 34 | AGGGGGCTGATGGTGAAAACG | 7_26 |
| 35 | GGGGGCTGATGGTGAAAACGA | 7_27 |
| 36 | GGGGGCTGATGGTAAAACGAA | 7_28 |

In some aspects, any one of SEQ ID NOs: 8-36 may include a 5′ and/or 3′ additional “nn” sequence, wherein n is any nucleic acid. In some aspects, the paired nucleotide strands can be provided as a double stranded composition of two annealed single-strands or a single self-annealed single stand, wherein a spacer of non-matching nucleotides is provided to allow for the single strand to fold back on itself without creating strain. A spacer may be of between about 3 to about 20 unmatching nucleotides in length, including 4, 5, 6, 7, 8, 9, 10, 11, 12, 13, 14, 15, 16, 17, 18, and 19 nucleotides in length. For example, the spacer can provide a hairpin loop in a single stranded nucleotide sequence.

In some aspects, the nucleotide sequences of the present disclosure can include a 5′ capped RNA, such as an RNA 7-methylguanosine cap or an RNA m 7 G cap. In some aspects, one stand of a two-stranded annealed complex is capped. In other aspects, both strands may be capped. In instances where the nucleotide sequence is a single-stranded self-annealing strand, the 5′ end of the strand map optionally be capped.

In some aspects, the nucleic acids as set forth herein may include synthetic and/or recombinant RNA nucleic acid sequences. Recombinant RNA sequences may be produced by introducing an expression vector or a cassette into a cell that includes a promoter operably linked to a nucleic acid sequence of RNA sequences as set forth herein to allow transcription through techniques understood in the art. Recombinant RNA can also be produced by introducing a viral vector, such as an adenovirus or adeno-associated virus, to a cell with the RNA nucleic acids sequences to be transcribed operably linked to a promoter. Synthetic nucleic acid sequences may similarly be produced using techniques known in the art, such as solid phase synthesis. In some aspects, the nucleic acids of the present disclosure may be produced by other means, such as in vitro transcription that can utilize a linear DNA with the sequence to be transcribed operably linked to a promoter. In either recombinant or synthetic methods, the RNA can be expressed as two separate strands or as a single, self-annealing strand. A self-annealing strand may include a spacer between complementary strands to provide a small hairpin loop, such that the strand is able to fold onto itself and self-anneal.

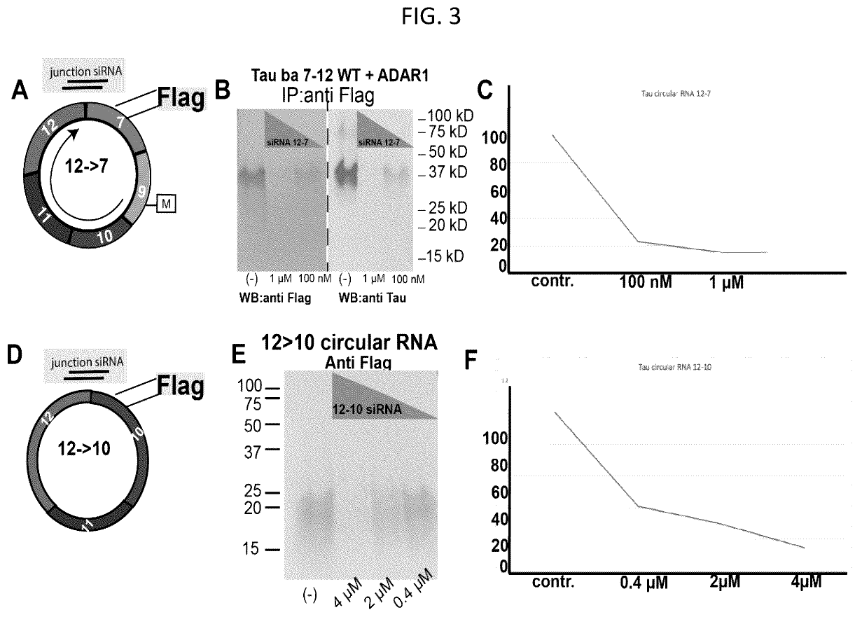

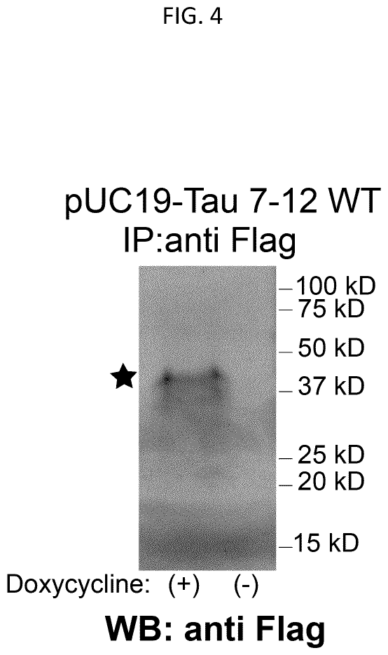

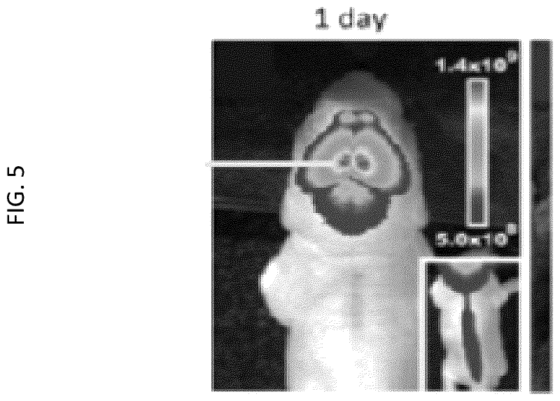

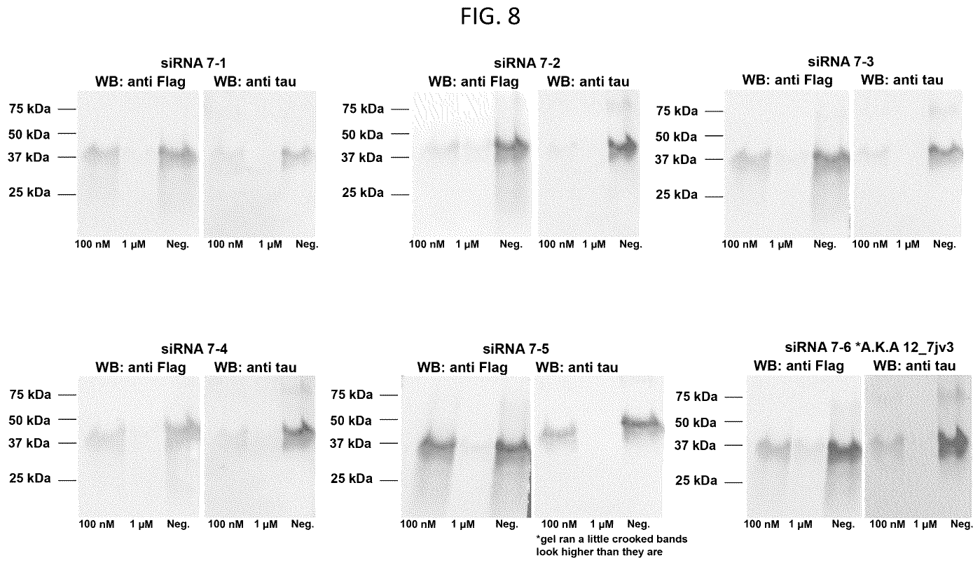

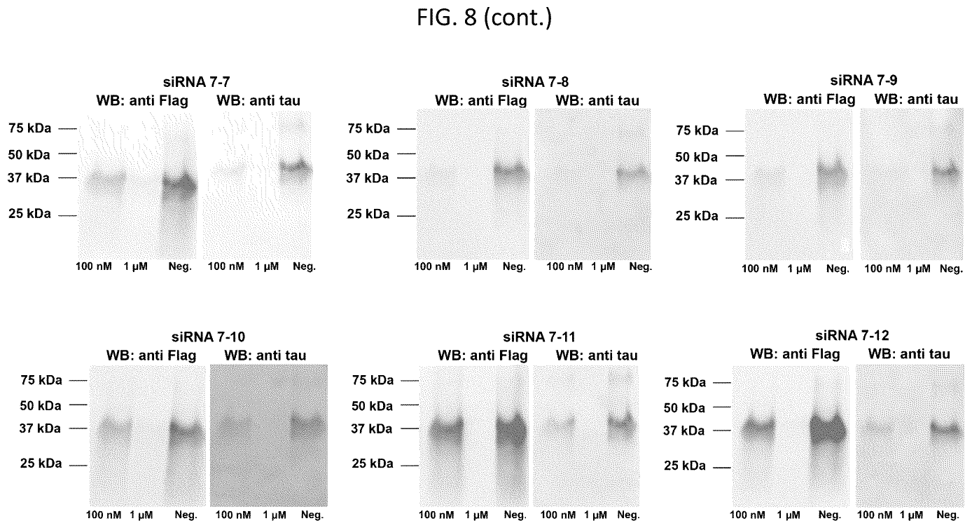

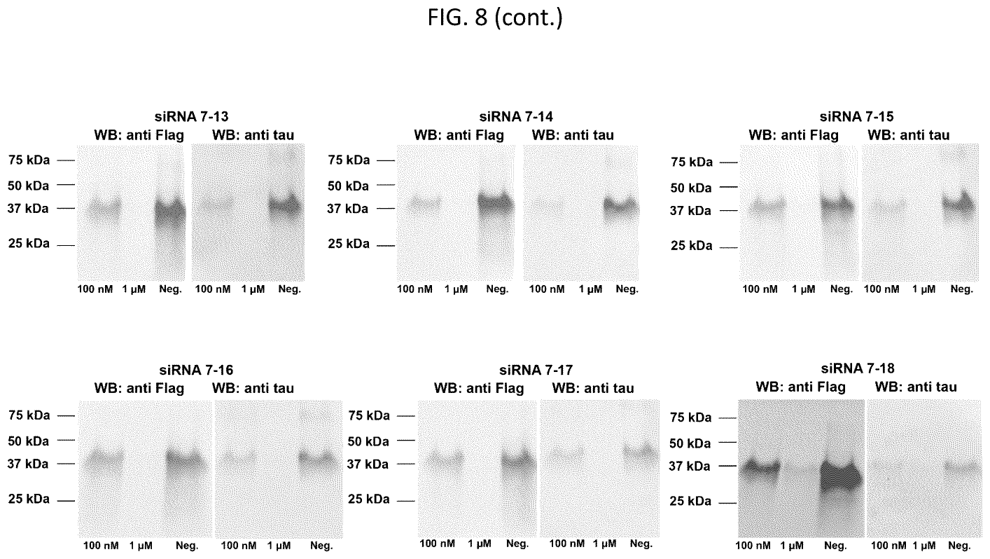

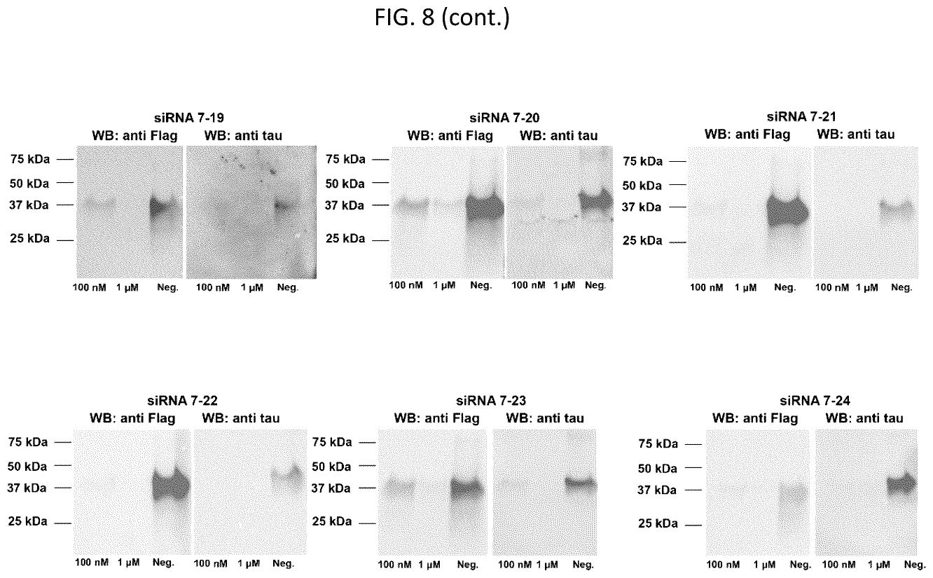

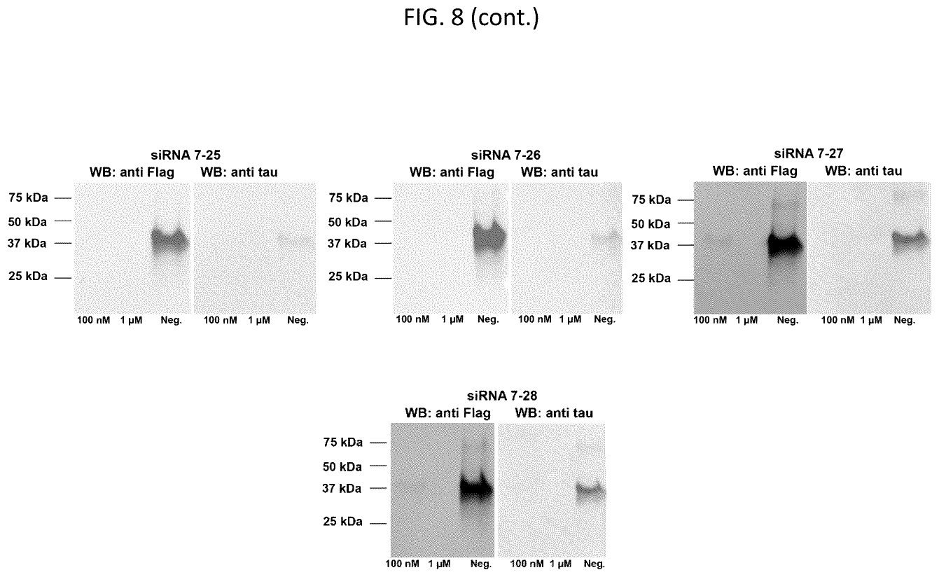

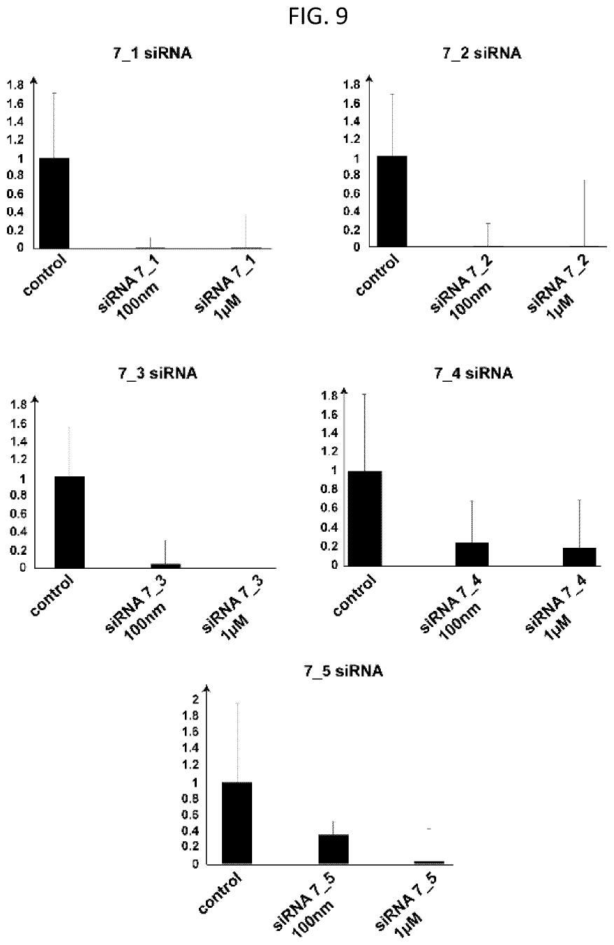

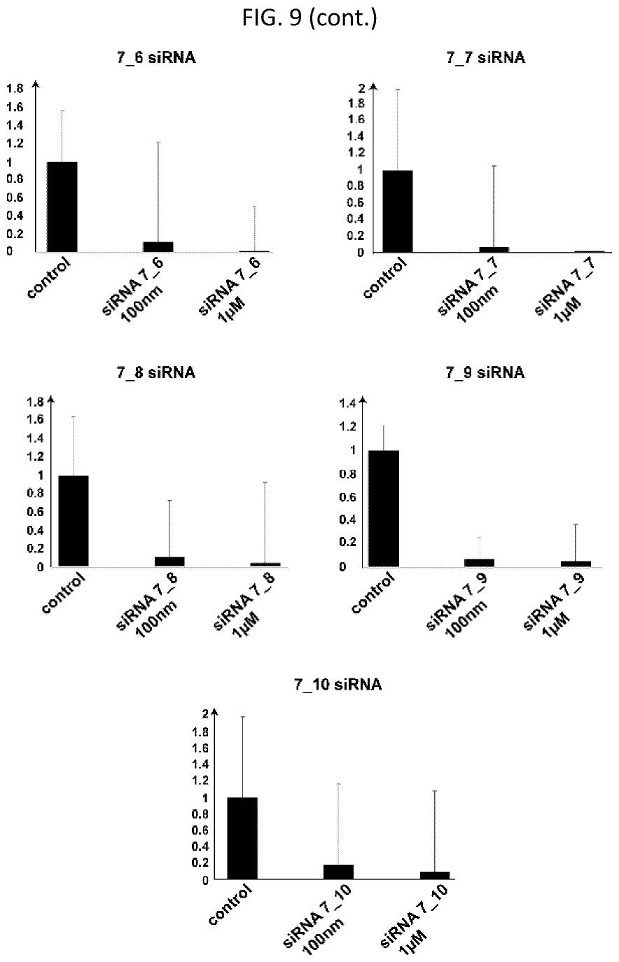

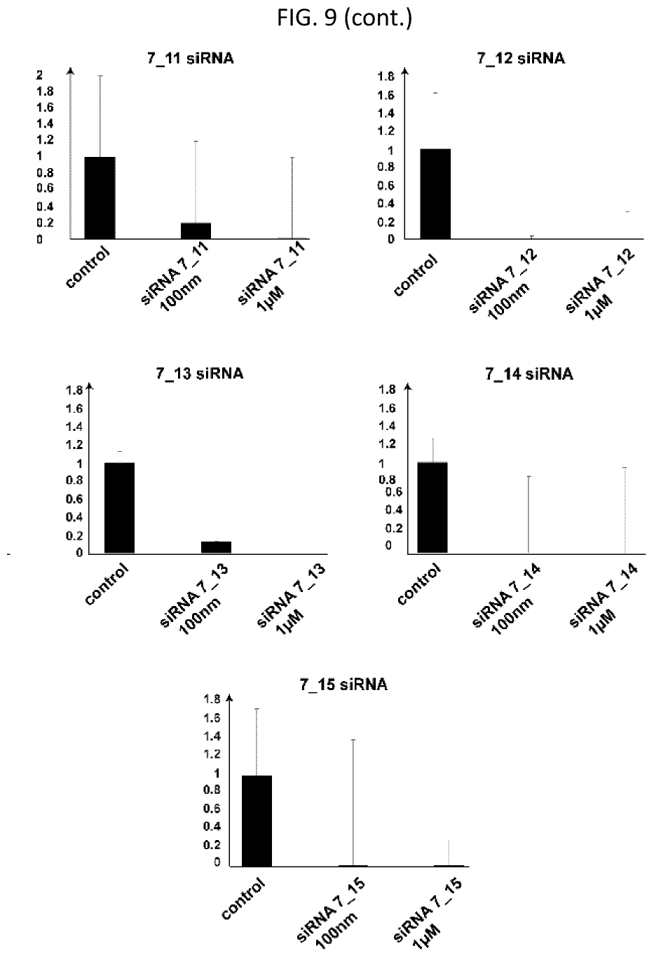

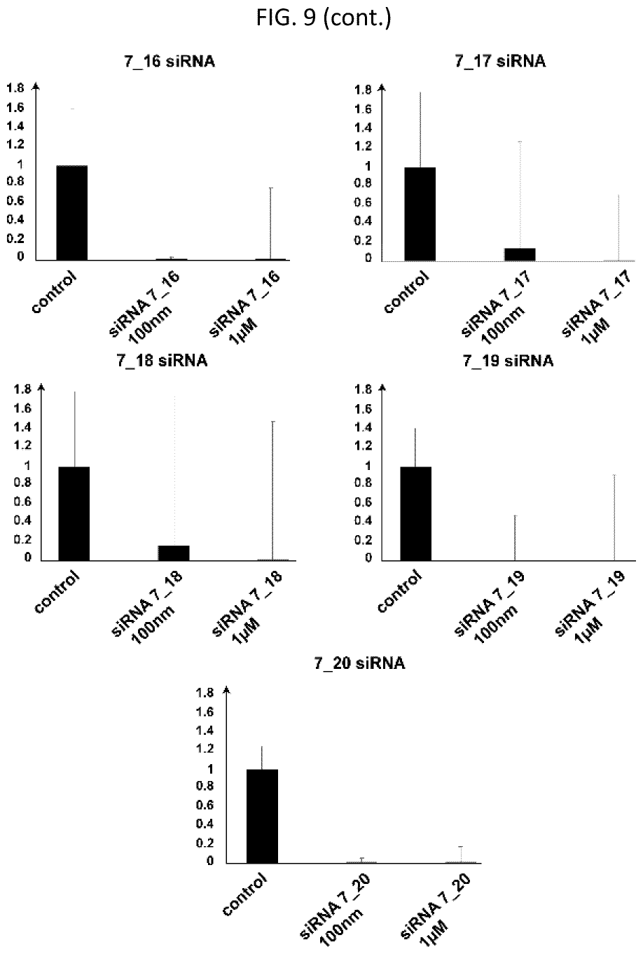

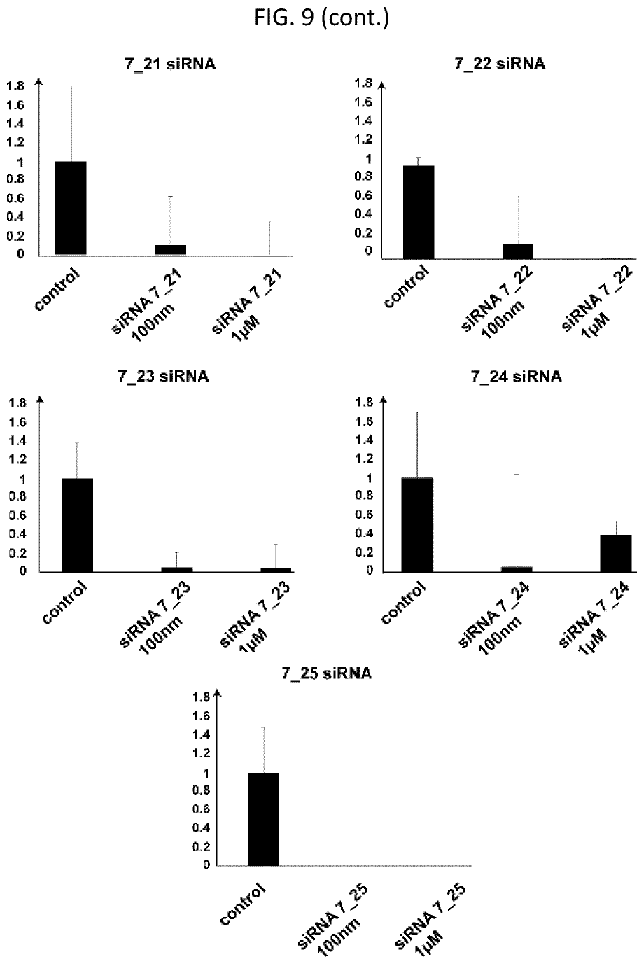

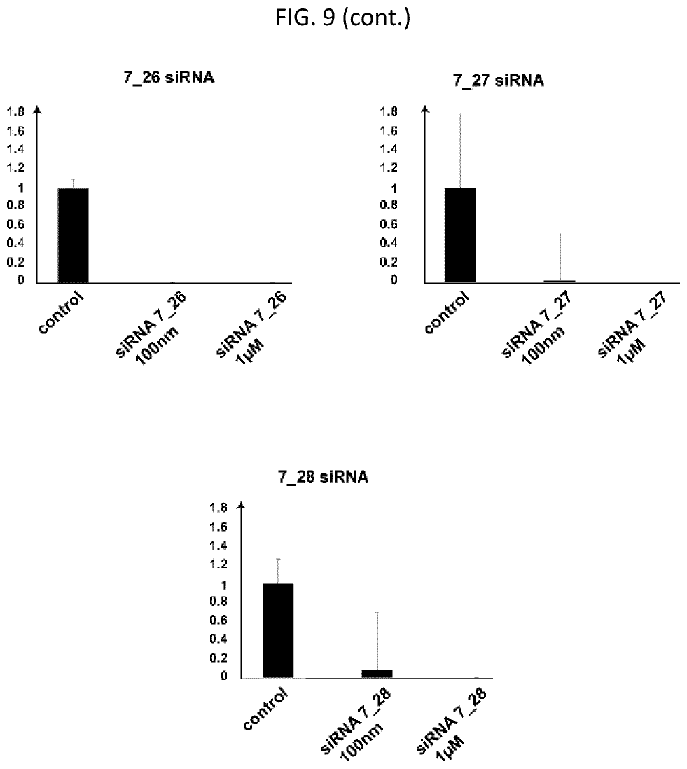

As set forth in the working examples and figures, a mouse model with a doxorubicin inducible promotor was established to provide a model to assess circRNA activity. To bring a coding sequence under a promoter's control, one positions the 5′ end of the translational initiation site of the reading frame generally between about 1 and 50 nucleotides downstream (i.e., 3′ of) of the promoter. The promoter stimulates transcription of the inserted fragment. FIGS. 4 and 5 confirm the incurable expression and the localization in the model's brain tissue. FIGS. 3A, 3B, and 3C shows an overview of siRNA design, expression, and inhibition with the noted 12->7 siRNA. FIGS. 3D, 3E, and 3F shows the same for a 12->10 siRNA. FIGS. 8 and 9 further confirm inhibition of the circRNA expression at the indicated concentrations added.

The data herein show that the siRNAs as set forth herein can remove tau circular RNA. The current siRNAs target the junctions as depicted herein with nanomolar dosing showing to be effective. Further evidence can be seen in PCT/US2022/074065 and Welden et al. (“RNA editing of microtubule associated protein tau circular RNAs promotes their translation and tau tangle formation” Nucleic Acids Res. (2022-12-0 50(22): 12979-12996) (both incorporated by reference herein in their entirety). The siRNA target a junction of a unique backsplice.

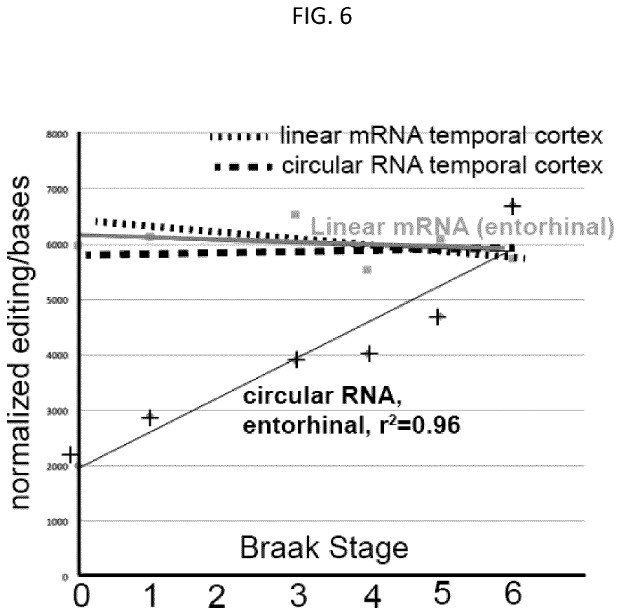

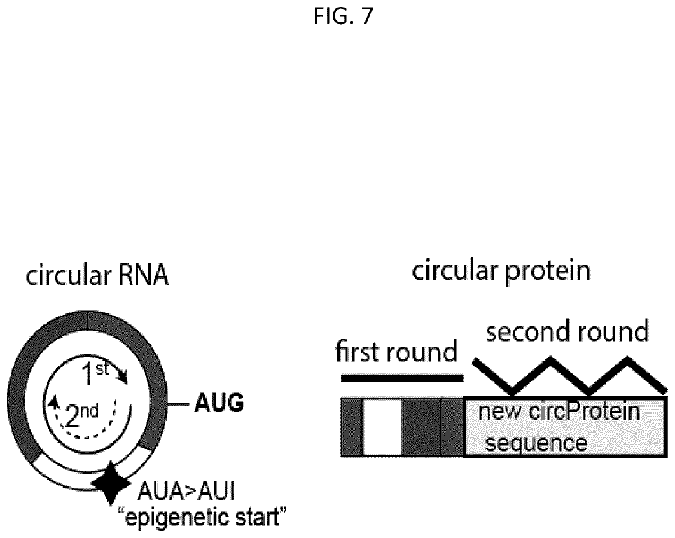

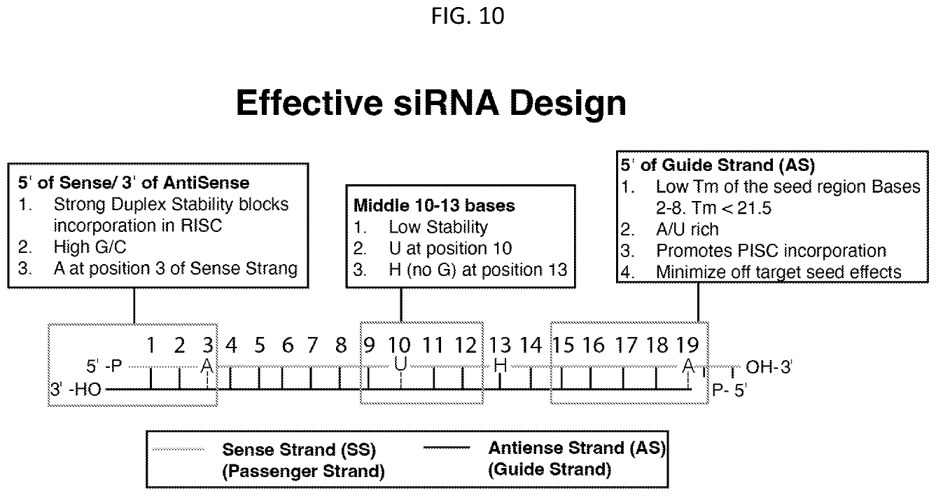

In some aspects, the siRNA can be further modified to improve stability, such as to resist and/or prevent nuclease degradation. As set forth in FIG. 6, circRNA expression correlates with the increase in Braak staging. Accordingly, reducing or silencing the circRNA activity can provide a treatment to decrease aggregates in brain tissue, as well as reduce NFTs therein. In some aspects, FIG. 10 outlines some proposed approaches for modifying the siRNA as set forth herein. In some aspects, the sense and/or sense strands may include 2′-O-methylation, 2′-O-ethylation, 2′-fluorination, 2′-desoxylation, and/or 5′-phosphorylation. In some aspects, the sense and/or antisense strands may include a mono- or di-phosphothionate. In some aspects, the siRNA molecules may be nucleoside modified through the incorporation of modified bases, such as pseudouridine, 1-methylpseudouridine, 5-methlycytidine, N6-methyl adenosine, 2-thio-uridine, and 5-methoxyuridine. Modifications may occur at either the 5′ terminus and/or the 3′ terminus, as well as at one or more internal nucletides therein. In some aspects, one or more of bases 5-18 of the sense and/or antisense strands of the siRNA are modified. In some aspects of the present disclosure, the nucleic acids as set forth herein may include one or more modified nucleic acid bases and/or a xeno nucleic acids. Such may include 5′ phosphorylation for recognition by the silencing complex, backbone modifications (phosphotriester substitutions and/or phosphorothioate/boranophosphate/phosphonoacetate linkages), sugar modifications, base modifications and/or lipid conjugations. For example, a nucleic acid base may be nucleoside modified through the incorporation of modified bases, such as pseudouridine, 1-methylpseudouridine, 5-methlycytidine, N6-methyl adenosine, 2-thio-uridine, and 5-methoxyuridine. Other modifications include 2′fluorination, 2′O-methylation, 2′O-ethylation, locked nucleic acids, C7 adenine or guanosine modifications, C5 uridine or cytosine modifications, incorporation of 2′ fluoro arabinose, incorporation of alkyl phosphonate nucleic acids, incorporation of 2′-deoxyxylnucleic acids, peptide or amino acid conjugated bases, and/or use of a phosphothionate or borano-phosphate moiety. See, e.g., Duffy et al. BMC Biol. 18, 112, (2020) and Chernikov et al. Front. Pharmacol. 10: 444 doi: 10.3389/fphar.2019.00444, 2019.

In some aspects, the present disclosure includes a vector for expressing the one or more strands of the siRNAs as set forth herein. The vector may include a promoter, such as an inducible promoter that drives transcription of the one or more strands of siRNA.

In some aspects, the siRNA of the instant disclosure include two separate strands that are hybridized or capable thereof to form the ds structure. In some aspects, the siRNA is a single strand that can fold back on itself, such as with a hairpin sequence separating the sense and antisense sequences.

In some aspects, the present disclosure includes pharmaceutical compositions for the delivery of the siRNA as set forth herein. In some aspects, the siRNA may be administered alone or in combination with a further therapeutic agent, an adjuvant, an excipient, and/or a pharmaceutical acceptable carrier. Administration may further be with a pharmaceutically acceptable carrier or excipient or diluent, which refers to a non-toxic accompanying salt, sugar, gum, coating or similar. Such are described in the most recent edition of Remington: The Science and Practice of Pharmacy (23^{rd} Ed., Academic Press, 2020).

In some aspects, the siRNA is formulated as a therapeutic for delivery to a subject, such as a human subject. In some aspects, the siRNA is formulated for oral, nasal, intramuscular, intravenous, rectal, sublingual, transdermal, ocular, and/or intraperitoneal delivery. Administration may occur when the compositions are included in a suspension, capsule or tablet, a delayed release network or implant. It will be appreciated that the compositions need to access the extracellular space within the central nervous system. Accordingly, while typical routes of administration, such as intravenous, oral, sublingual, and other systemic routes are contemplated, it is further contemplated that for effective delivery to the central nervous system of a subject in vivo, such as an animal subject including a human, that intrathecal delivery, such as through spinal injection, may be necessary. Other routes of administration may include nasal delivery to the central nervous system (CNS) and intraventricular delivery, either through injection or through the use of an osmotic pump.

In some aspects, the siRNA are formulate with one or more lipid vehicles to assist or promote cellular uptake in vivo. Such may include a “helper” lipid, such as 1,2-dioleoyl-3-trimethylammoniumpropane (DOTAP) and/or dioleoylphosphatidylethanolamine (DOPE) and/or lipofectamine and/or dioleoylphosphatidylcholine (DOPC) and/or phosphatidylethanolamine (dioleoyl PE) and/or 3β-[N—(N′,N′-dimethylaminoethane)-carbamoyl]-cholesterol (DC-Chol). Such may include lipid nanoparticles (“LNP”) of an ionizable lipid (usually marked by three sections of an amine head, a linker and a hydrophobic tail, e.g. heptatriaconta-6,9,28,31-tetraen-19-yl 4-(dimethylamino)butanoate (DLin-MC3-DMA or MC3), DLinDMA, and DLin-KC2-DMA). In some aspects, the LNP may include an ionizable lipid, a polyethylene glycol and a cholesterol. In further aspects, the LNP may include a combination of an ionizable lipid with polyethylene glycol (PEG), cholesterol and/or distearoyl phosphocholine. Such may include a polymer or a polymer modified with fatty chains or a polymethacrylate with amine-bearing side chains or a polyaspartamide with oligoaminoethylene side chains or a poly(beta-amino) ester (PBAE). In some aspects, the composition may include a dendrimer. In some aspects, the compositions may include a cell-penetrating peptide and/or a carrier protein. In some aspects, the compositions may include siRNA encapsulated in a lipid or lipid-like nanoparticle. Such nanoparticles may include an ionizable lipid, a cholesterol, a polyethylene glycol and/or a helper lipid, such as DOTAP, DOPE, DOPC and/or dioleoyl PE.

The protein products of circular RNAs as Alzheimer's markers as well as provide targets for novel therapeutics. Increases in RNA editing may be present in AD in the entorhinal cortex. In some instances there may be an increase in translation of circular RNAs. Due to a rolling translation mechanism, the circRNAs express new peptides.

In some aspects, the present disclosure concerns antibodies or fragments thereof that bind to the peptides produced by the circRNAs as set forth herein. In some aspects, the antibodies bind to a peptide as set forth in one of SEQ ID NOs: 8-36. It will be appreciated that the antibody may be a monoclonal or a polyclonal antibody. The antibody may be of an immunoglobulin (Ig) selected from IgG, IgA, IgM, and/or IgE. The antibody may a single heavy chain or of a heavy and light chain. Fragments may include portions of the antibody that retain the ability to bind the circRNA peptides, including three complementary-determining regions (CDRs) within a heavy chain or six CDRs shared between heavy and light chains. Fragments may include VH domains, VL domains, VHH domains, V-NAR domains, V domains, Fab fragments, Fab₂ fragments, scFv fragments, diabodies, miniboides, tribodies, tetrabodies, and the like understood in the art.

In some aspects, the present disclosure concerns identifying the presence of the circRNAs as set forth herein correlates to the onset and/or progression of Alzheimer's through hybridization of one or more of SEQ ID NOs: 8-19 and/or 29-36. In some aspects, the methods of the present disclosure concern detecting or assaying for the presence of one or more of the circRNAs. In some aspects, the methods include obtaining a sample from the subject. In some aspects, the sample is of a fluid from the subject, such as cerebrospinal fluid and/or blood or plasma. In some aspects, the methods include assaying the sample for the presence of one or more of the circRNAs as set forth herein. In some aspects, the methods include contacting or administering to the sample one or more of the nucleotides (such as one or more of SEQ ID Nos: 8-19 and 29-36) disclosed herein to assay for hybridization to a nucleotide in the sample. Determining whether a nucleotide hybridizes or binds to a nucleotide sequence in the sample or determining whether an antibody binds to an antigenic protein or peptide within the sample may require secondary or additional reagents to confirm the reaction, as are understood in the art with techniques such as Northern blotting, Southern blotting, Western blotting, immunoprecipitation, immunostaining, ELISA, colorometric responses, fluorescent response, radiolabeling, conjugated antibodies, labeled antibodies, labeled nucleotides, and the like.

In some aspects, the determining of the presence of one or more of the circRNAs as set forth herein is indicative of the onset or progression of Alzheimer's disease and/or neurodegeneration in the subject. In some aspects, such results may allow for a course of treatment for the subject. In some aspects, such treatment may include administration to the subject of one or more siRNA, dsRNA or antisense thereof as described herein to reduce or lower one or more circRNA and/or protein/peptide expressed thereby. In some aspects, the methods may include assaying the subject over a period of time. As described herein, circRNA levels and/or proteins/peptides expressed thereby can correlate to disease progression. In some aspects, one may determine the Braak stage of the patient by determining the concentration and/or amount of circRNA in the subject or a sample obtained therefrom. In some aspects, a Braak stage may be determined from one assayed sample.

In some aspects, the present disclosure concerns methods of using the siRNA as set forth herein. In some aspects, the methods can alleviate or clear MAPT protein aggregates. In some aspects, the methods can alleviate symptoms of Alzheimer's Disease (AD). In some aspects, the methods can be to treat AD. It will be understood=d that the methods include administering to a subject in need, such as an AD subject the siRNA as set forth herein, either alone or as part of a composition as set forth herein. It will also be understood that administration of the siRNA as set forth herein may prevent MAPT aggregates from forming and/or from progressing in growth.

Further aspects and advantages of this disclosure are provided in the following section, which should be considered as illustrative only.

### EXAMPLES

A graft system was to create a model for AD. As the circular RNAs are human-specific, adaptations were needed to manifest their presence and translation in other animals for study. A human iPS cell line was generated with a doxycycline inducible tau circRNA (circular RNA). This allowed for the inducible expression of cirTau RNAs through an inducible promoter in the iPS cells. This then allows for predifferentiation into neuronal precursor cells that can be grafted into brain tissue within a mouse model (see, FIGS. 4 and 5). The formation of NFTs (with and without circRNA expression) can then be observed. Similarly, models can be prepare using traditional mouse models with circularization being driven by mouse RNA structures.

siRNA as set forth in SEQ ID NOs: 8-36 were administered to the animals and the subsequent expression of the cirRNA assessed. FIGS. 8 and 9 confirm that the siRNAs as set forth herein successfully silence the presence of the circRNA of the 12->7 backsplice, as well as quench expression of the corresponding protein.

Various modifications of the present disclosure, in addition to those shown and described herein, will be apparent to those skilled in the art of the above description. Such modifications are also intended to fall within the scope of the appended claims.

It is appreciated that all reagents are obtainable by sources known in the art unless otherwise specified.

It is also to be understood that this disclosure is not limited to the specific aspects and methods described herein, as specific components and/or conditions may, of course, vary. Furthermore, the terminology used herein is used only for the purpose of describing particular aspects of the present disclosure and is not intended to be limiting in any way. It will be also understood that, although the terms “first,” “second,” “third” etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, “a first element,” “component,” “region,” “layer,” or “section” discussed below could be termed a second (or other) element, component, region, layer, or section without departing from the teachings herein. Similarly, as used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

Reference is made in detail to exemplary compositions, aspects and methods of the present disclosure, which constitute the best modes of practicing the disclosure presently known to the inventors. The drawings are not necessarily to scale. However, it is to be understood that the disclosed aspects are merely exemplary of the disclosure that may be embodied in various and alternative forms. Therefore, specific details disclosed herein are not to be interpreted as limiting, but merely as a representative basis for any aspect of the disclosure and/or as a representative basis for teaching one skilled in the art to variously employ the present disclosure.

Patents, publications, and applications mentioned in the specification are indicative of the levels of those skilled in the art to which the disclosure pertains. These patents, publications, and applications are incorporated herein by reference to the same extent as if each individual patent, publication, or application was specifically and individually incorporated herein by reference.

The foregoing description is illustrative of particular embodiments of the disclosure, but is not meant to be a limitation upon the practice thereof. The following claims, including all equivalents thereof, are intended to define the scope of the disclosure.

We claim:

1. An isolated double stranded (ds) silencing ribonucleic acid (siRNA) comprising a nucleotide sequence comprised of a 3′ terminal nucleic acid from exon 12 of the MAPT gene fused to a 5′ terminal nucleic acid from either exon 7 of the MAPT gene or exon 10 of the MAPT gene.

2. The isolated ds siRNA of claim 1, wherein the nucleotide sequence comprises 21 nucleotides in length, with up to 20 nucleotides being derived from the 3′ end of exon 12 of the MAPT gene.

3. The isolated ds siRNA of claim 1, wherein the nucleotide sequence comprises 21 nucleotides in length, with up to 20 nucleotides being derived from the 5′ end of exon 7 of the MAPT gene.

4. The isolated ds siRNA of claim 1, wherein the nucleotide sequence comprises 21 nucleotides in length, with up to 20 nucleotides being derived from the 5′ end of exon 10 of the MAPT gene.

5. The isolated ds siRNA of claim 1, wherein the nucleotide sequence comprises 21 contiguous nucleotides in length from SEQ ID NO: 28.

6. The isolated ds siRNA of claim 5, wherein the nucleotide sequence comprises a sequence selected from SEQ ID NOs: 8-19 and 29-36.

7. The isolated ds siRNA of claim 1, wherein the wherein one or more nucleic acids are modified.

8. The isolated ds siRNA of claim 7, wherein the modification includes incorporation of 2′-O-methylation, 2′-O-ethylation, 2′-fluorination, 2′-desoxylation, and/or 5′-phosphorylation, mono-phosphothionate, or di-phosphothionate.

9. The isolated ds siRNA of claim 1, wherein the double strands are two separate annealed strands.

10. The isolated ds siRNA of claim 1, wherein the double strands are a self-annealed single strand.

11. The isolated ds siRNA of claim 10, further comprising a spacer of 3 to about 20 unmatching nucleotides in length between a sense portion and an antisense portion.

12. The isolated ds siRNA of claim 1, wherein the double strands are a self-annealed single strand.

13. A lipid nanoparticle comprising the isolated ds siRNA of claim 1 and a lipid membrane.

14. A method of treating or alleviating formation of neurofibrillary tangles comprising administering the isolated ds siRNA of claim 1 to a subject.

15. The method of claim 14, wherein the ds siRNA is administered nasally and/or by intrathecal injection.

16. A method to treat aggregates of MAPT proteins in brain tissue, comprising administering the isolated ds siRNA of claim 1 to a subject.

17. The method of claim 16, wherein the ds siRNA is administered nasally and/or by intrathecal injection.

18. A method for alleviating Alzheimer's Disease (AD) comprising administering to a subject with AD the isolated ds siRNA of claim 1.

19. A method for treating AD comprising administering to a subject with AD the isolated ds siRNA of claim 1.


SEQUENCE LISTING (36 sequences)

SEQ ID NO: 1 - RNA, 681 nt, synthetic construct
  source 1..681: mol_type = other RNA
ggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaag

SEQ ID NO: 2 - RNA, 747 nt, synthetic construct
  source 1..747: mol_type = other RNA
ggggctgatggtaaaacgaaggactacaaagaccatgacggtgattataaagatcatgacatcgattacaaggatgacgatgacaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaag

SEQ ID NO: 3 - RNA, 21 nt, synthetic construct
  source 1..21: mol_type = other RNA
aggaaataaaaagggggcttt

SEQ ID NO: 4 - RNA, 21 nt, synthetic construct
  source 1..21: mol_type = other RNA
agccccctttttatttcctcc

SEQ ID NO: 5 - RNA, 20 nt, synthetic construct
  source 1..20: mol_type = other RNA
ggggctgatggtgactacaa

SEQ ID NO: 6 - RNA, 21 nt, synthetic construct
  source 1..21: mol_type = other RNA
ggcggaggaaataaaaaggtt

SEQ ID NO: 7 - RNA, 40 nt, synthetic construct
  source 1..40: mol_type = other RNA
ctggcggaggaaataaaaagggggctgatggtgactacaa

SEQ ID NO: 8 - RNA, 21 nt, synthetic construct
  source 1..21: mol_type = other RNA
ctggcggaggaaataaaaagg

SEQ ID NO: 9 - RNA, 21 nt, synthetic construct
  source 1..21: mol_type = other RNA
tggcggaggaaataaaaaggg

SEQ ID NO: 10 - RNA, 21 nt, synthetic construct
  source 1..21: mol_type = other RNA
ggcggaggaaataaaaagggg

SEQ ID NO: 11 - RNA, 21 nt, synthetic construct
  source 1..21: mol_type = other RNA
gcggaggaaataaaaaggggg

SEQ ID NO: 12 - RNA, 21 nt, synthetic construct
  source 1..21: mol_type = other RNA
cggaggaaataaaaagggggc

SEQ ID NO: 13 - RNA, 21 nt, synthetic construct
  source 1..21: mol_type = other RNA
ggaggaaataaaaagggggct

SEQ ID NO: 14 - RNA, 21 nt, synthetic construct
  source 1..21: mol_type = other RNA
gaggaaataaaaagggggctg

SEQ ID NO: 15 - RNA, 21 nt, synthetic construct
  source 1..21: mol_type = other RNA
aggaaataaaaagggggctga

SEQ ID NO: 16 - RNA, 21 nt, synthetic construct
  source 1..21: mol_type = other RNA
ggaaataaaaagggggctgat

SEQ ID NO: 17 - RNA, 21 nt, synthetic construct
  source 1..21: mol_type = other RNA
gaaataaaaagggggctgatg

SEQ ID NO: 18 - RNA, 21 nt, synthetic construct
  source 1..21: mol_type = other RNA
aaataaaaagggggctgatgg

SEQ ID NO: 19 - RNA, 21 nt, synthetic construct
  source 1..21: mol_type = other RNA
aataaaaagggggctgatggt

SEQ ID NO: 20 - RNA, 21 nt, synthetic construct
  source 1..21: mol_type = other RNA
ataaaaagggggctgatggtg

SEQ ID NO: 21 - RNA, 21 nt, synthetic construct
  source 1..21: mol_type = other RNA
taaaaagggggctgatggtga

SEQ ID NO: 22 - RNA, 21 nt, synthetic construct
  source 1..21: mol_type = other RNA
aaaaagggggctgatggtgac

SEQ ID NO: 23 - RNA, 21 nt, synthetic construct
  source 1..21: mol_type = other RNA
aaaagggggctgatggtgact

SEQ ID NO: 24 - RNA, 21 nt, synthetic construct
  source 1..21: mol_type = other RNA
aaagggggctgatggtgacta

SEQ ID NO: 25 - RNA, 21 nt, synthetic construct
  source 1..21: mol_type = other RNA
aagggggctgatggtgactac

SEQ ID NO: 26 - RNA, 21 nt, synthetic construct
  source 1..21: mol_type = other RNA
agggggctgatggtgactaca

SEQ ID NO: 27 - RNA, 21 nt, synthetic construct
  source 1..21: mol_type = other RNA
gggggctgatggtgactacaa

SEQ ID NO: 28 - RNA, 40 nt, synthetic construct
  source 1..40: mol_type = other RNA
ctggcggaggaaataaaaagggggctgatggtaaaacgaa

SEQ ID NO: 29 - RNA, 21 nt, synthetic construct
  source 1..21: mol_type = other RNA
ataaaaagggggctgatggta

SEQ ID NO: 30 - RNA, 21 nt, synthetic construct
  source 1..21: mol_type = other RNA
taaaaagggggctgatggtaa

SEQ ID NO: 31 - RNA, 21 nt, synthetic construct
  source 1..21: mol_type = other RNA
aaaaagggggctgatggtaaa

SEQ ID NO: 32 - RNA, 21 nt, synthetic construct
  source 1..21: mol_type = other RNA
aaaagggggctgatggtaaaa

SEQ ID NO: 33 - RNA, 21 nt, synthetic construct
  source 1..21: mol_type = other RNA
aaagggggctgatggtaaaac

SEQ ID NO: 34 - RNA, 21 nt, synthetic construct
  source 1..21: mol_type = other RNA
aagggggctgatggtaaaacg

SEQ ID NO: 35 - RNA, 21 nt, synthetic construct
  source 1..21: mol_type = other RNA
agggggctgatggtaaaacga

SEQ ID NO: 36 - RNA, 21 nt, synthetic construct
  source 1..21: mol_type = other RNA
gggggctgatggtaaaacgaa